(19) United States
(12) Patent Application Publication
(10) Pub. No.: US 2011/0172204 A1
(43) Pub. Date: Jul. 14, 2011

(54) COMPOUNDS AND THEIR USE

(75) Inventors: Parminder Kaur Pooni, Cambridge (GB); Kevin John Merchant, Cambridge (GB); William John Buffham, Cambridge (GB)

(73) Assignee: TAKEDA PHARMACEUTICAL COMPANY LIMITED, Osaka (JP)

(21) Appl. No.: 12/986,391

(22) Filed: Jan. 7, 2011

(30) Foreign Application Priority Data
Jan. 8, 2010 (GB) 1000305.1
Oct. 25, 2010 (GB) 1018028.9

(51) Int. Cl. A61K 31/55; C07D 403/12; C07D 403/04; C07D 403/14; C07D 401/14; A61P 25/00; A61P 25/28; A61P 3/04; A61P 25/18; A61P 25/08

(52) U.S. Cl. 514/210.21; 540/594; 514/217.01; 544/332

(57) ABSTRACT

A compound of the formula:

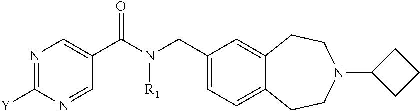

wherein:
R₁ represents C₁₋₆ alkyl or H; Y represents —NR₂R₃ as depicted in formula (B), or a ring of formula (A)

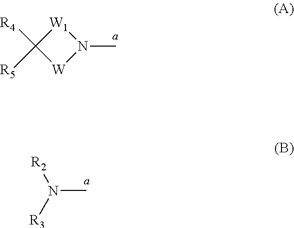

wherein ^{a} represents the point of attachment to the pyrimidinyl ring;
R₂ represents C₁₋₄alkyl substituted by C₁₋₃ alkoxy; R₃ represents C₁₋₄ alkyl; W represents —(CH₂)ₙ—; W₁ represents —(CH₂)ₚ—; n represents 1 or 2 or 3; p represents 1 or 2; R₄ represents C₁₋₄ alkoxy, C₁₋₆ alkyl or halogen; and R₅ represents halogen or H, provided that, when R₄ represents halogen, R₅ is not H, or a pharmaceutically acceptable salt thereof. The compounds of the invention have been found to modulate the histamine H3 receptor.

The present invention relates to compounds and their uses, and in particular to compounds having a benzazepine scaffold and their therapeutic use in the treatment or prevention of conditions having an association with the histamine H3 receptor.

The H3 receptor was first identified pharmacologically in 1983 as an autoreceptor that regulates the production of histamine (1). The receptor was later cloned in 1999 (2). It is a constitutively active G protein-coupled receptor that is expressed predominantly in the central nervous system (CNS) and modulates a variety of CNS functions both centrally and peripherally. It is expressed on the presynaptic terminals of CNS neurones and acts as a negative modulator of release of neurotransmitters such as histamine, acetylcholine, norepinephrine, serotonin and dopamine (3). Consequently, the ability of the H3 receptor to regulate the release of a wide range of neurotransmitters has fuelled research into the development of antagonists/inverse agonists which have potential in behavioural and physiological conditions, for example CNS disorders such as narcolepsy, disorders of wakefulness, cognition or attention, pain and in suppression of food intake.

Histaminergic neurones are located in the tuberomammillary nucleus of the posterior hypothalamus and project their axons into brain regions including the hypothalamus, thalamus, cerebral cortex, amygdala, and septum. Activity of histaminergic neurons is closely linked with the sleep/wake cycle and numerous reports in the literature have established that the H3 receptor plays a role in cognition and sleep/wake related processes, based on studies with known H3 receptor antagonists and their effects in animal models (4, 5, 6). H3 antagonist compound A-349821 is currently in preclinical development and has been shown to demonstrate cognition-enhancing effects in the rat (7).

The histaminergic system is one of the targets of leptin signalling in the hypothalamus. Known H3 antagonist clobenpropit increases histamine release in the hypothalamus of mice and has the effect of reducing energy intake in both lean and obese mice (8). The role of the H3 receptor in obesity has been further substantiated through studies with antagonists thioperamide and ciproxifan and more recently with non-imidazole compounds (10).

The non-selective antagonist thioperamide has an antinociceptive effect in a number of acute pain models (11). H3 antagonists have been suggested for the treatment of neuropathic pain (12). In addition GSK207040 and GSK334429 are selective non-imidazole H3 antagonist compounds that display high affinity for both rat and human H3 receptors. Both compounds reduced tactile allodynia in the rat, suggesting H3 antagonists have therapeutic potential in the treatment of neuropathic pain (13).

In an attempt to identify compounds with improved drug-like properties, non-imidazole compounds have been at the forefront of research, for example A-349821 (7) and GSK207040/GSK334429 (13). ABT-239 is currently being investigated for use in attention deficit hyperactivity disorder, Alzheimer's Disease and schizophrenia (14).

WO05/123723, WO06/018260 and WO05/058837 disclose H3 antagonist benzazepine derivatives claimed to be useful in the treatment of neurological and psychiatric disorders. WO05/058328 discloses dopamine D3 receptor benzazepine derivatives claimed to be useful in the treatment of CNS disorders such as schizophrenia and depression. WO02/40471 also discloses benzazepine derivatives useful as modulators of the dopamine D3 receptor. US2003/0158177 discloses melanin-concentrating hormone antagonists claimed to be useful in the treatment of obesity.

There exists a clinical need to generate further classes of H3 antagonist and/or inverse agonist compounds that demonstrate improved drug-like properties (9).

In accordance with a first aspect of the present invention, there is provided a compound having the Formula (1):

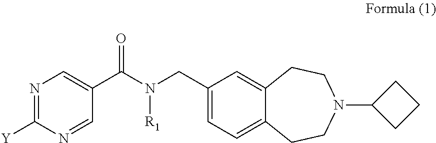

wherein:
R₁ represents C₁₋₆ alkyl or H;
Y represents —NR₂R₃ as depicted in formula (B), or a ring of formula (A)

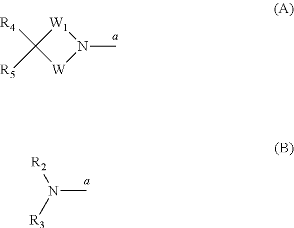

wherein ^{a} represents the point of attachment to the pyrimidinyl ring;
R₂ represents C₁₋₄ alkyl substituted by C₁₋₃ alkoxy;
R₃ represents C₁₋₄ alkyl;
W represents —(CH₂)ₙ—;
W₁ represents —(CH₂)ₚ—;
n represents 1, 2 or 3;
p represents 1 or 2;
R₄ represents C₁₋₄ alkoxy, C₁₋₆ alkyl or halogen; and
R₅ represents halogen or H;
provided that, when R₄ represents halogen, R₅ is not H,
or a pharmaceutically acceptable salt thereof.

The compounds of the invention have been found to modulate the histamine H3 receptor. In particular, the compounds possess antagonist or inverse agonist properties at this receptor. Based on the high affinity for the receptor, the compounds may have the potential to display useful selectivity for the H3 receptor. Compounds of the invention have been found to display properties suggestive of blood brain barrier permeability, rendering them potentially suitable for the treatment of CNS disorders.

In the compounds of the invention as represented by formula (1) and the more detailed description hereinafter certain of the general terms used in relation to substituents are to be understood to include the following atoms or groups unless otherwise specified.

The term ‘C_{x-y} alkyl’ as used herein refers to a linear or branched saturated hydrocarbon group containing from x to y carbon atoms. For example, C₁₋₆ alkyl refers to a linear or branched saturated hydrocarbon group containing from 1 to 6 carbon atoms. Examples of C₁₋₆ alkyl groups include methyl, ethyl, n-propyl, isopropyl, n-butyl, isobutyl, sec-butyl, tert butyl, n-pentyl, isopentyl, neopentyl and hexyl.

The term ‘C_{x-y} alkoxy’ as used herein refers to an —O—C_{x-y} alkyl group wherein C_{x-y} alkyl is as defined herein. Examples of such groups include methoxy, ethoxy, propoxy and butoxy.

The term ‘halogen’ as used herein refers to a fluorine, chlorine, bromine or iodine atom, unless otherwise specified. Typically, a fluorine is employed.

‘Pharmaceutically acceptable salts’ of compounds of Formula I of the present invention include salts with inorganic bases, salts with organic bases, salts with inorganic acids, salts with organic acids and salts with basic or acidic amino acids. Salts with acids may, in particular, be employed in some instances. In particular, ‘pharmaceutically acceptable salts’ of compounds of Formula (1) of the present invention include but are not limited to acid addition salts (for example, phosphates, nitrates, sulphates, borates acetates, maleates, citrates, fumarates, succinates, methanesulfonates, benzoates, salicylates and hydrohalides), and salts of amino acids (such as glycine, alanine, valine, leucine, isoleucine, cysteine, methionine, proline). Further pharmaceutically acceptable salts include quaternary ammonium salts of the compounds of formula I.

Compounds of formula (1) and their salts may be in the form of a solvate, which is included in the scope of the invention. Such solvates may be formed with common organic solvents, including but not limited to, alcoholic solvents e.g. methanol, ethanol or isopropanol.

The compound of Formula I of the present invention may be in either hydrate or non-hydrate form. General methods for the preparation of salts are well known to the person skilled in the art. Pharmaceutical acceptability of salts will depend on a variety of factors, including formulation processing characteristics and in vivo behaviour, and the skilled person would readily be able to assess such factors having regard to the present disclosure.

Where compounds of the invention exist in different enantiomeric and/or diastereoisomeric forms (including geometric isomerism about a double bond), these compounds may be prepared as isomeric mixtures or racemates, although the invention relates to all such enantiomers or isomers, whether present in an optically pure form or as mixtures with other isomers. Individual enantiomers or isomers may be obtained by methods known in the art, such as optical resolution of products or intermediates (for example chiral chromatographic separation (e.g. chiral HPLC)), or an enantiomeric synthesis approach. Similarly, where compounds of the invention may exist as alternative tautomeric forms (e.g. keto/enol, amide/imidic acid), the invention relates to the individual tautomers in isolation, and to mixtures of the tautomers in all proportions.

In certain embodiments, the compounds of the invention bear one or more radiolabels. Such radio labels may be introduced by using radio label-containing reagents in the synthesis of the compounds of formula 1, or may be introduced by coupling the compounds of formula 1 to chelating moieties capable of binding to a radioactive metal atom. Such radio labeled versions of the compounds may be used, for example, in diagnostic imaging studies.

In certain embodiments of the first aspect of the invention, R₁ is C₁₋₆ alkyl (e.g. methyl, ethyl, propyl or isopropyl).

In particular embodiments, R₁ represents methyl or ethyl, and especially methyl.

In alternative embodiments, R₁ represents methyl or H. In such instances, R₁ may in particular represent H.

In certain particular embodiments Y represents a ring of formula (A).

In some embodiments, n represents 1. In other embodiments, n represents 2. In further embodiments, n represents 3. In particular embodiments, n represents 1 or 2. In further particular embodiments, when p represents 2, n represents 1 or 2.

In some embodiments p represents 1, i.e. W₁ represents —CH₂—. In other embodiments p represents 2.

In certain particular embodiments p represents 1 and n represents 1. In other particular embodiments p represents 1 and n represents 2. In further particular embodiment p represents 2 and n represents 2.

In compounds of the invention, R₂ represents C₁₋₄ alkyl (e.g. ethyl or propyl) substituted by C₁₋₃ alkoxy (e.g. methoxy). In certain embodiments, R₂ represents methoxypropyl or methoxyethyl. In such instances, R₂ may in particular represent methoxypropyl, typically 2-methoxypropyl.

In compounds of the invention, R₃ represents C₁₋₄ alkyl (e.g. methyl or ethyl). In certain embodiments, embodiment R₃ represents methyl.

In particular embodiments, R₂ represents C₁₋₄ alkyl substituted by C₁₋₃ alkoxy, and R₃ represents methyl.

In compounds of the invention, R₅ represents H, or halogen (e.g. F, Cl). In certain embodiments, R₅ represents H or F. In such instances, R₅ may in particular represent H (except when R4 is halogen).

In compounds of the invention, R₄ represents C₁₋₄ alkoxy (e.g. methoxy, ethoxy, propoxy), C₁₋₆alkyl (e.g. methyl, ethyl, propyl or isopropyl) or halogen (e.g. F or Cl).

In particular embodiments, R₄ represents C₁₋₄ alkoxy.

In certain embodiments, R₄ represents methoxy, ethoxy, or F. In particular embodiments, R₄ represents methoxy or F. In more particular embodiments, R₄ represents methoxy.

Particular embodiments of the first aspect of the invention include compounds wherein R₁ is H, n is 2, p is 1, R₄ is methoxy and R₅ is H.

Further particular embodiments of the first aspect of the invention include compounds wherein R₁ is H, n is 1, p is 1, R₄ is methoxy and R₅ is H.

In some embodiments, compounds of the invention are in the form of the (S) enantiomers. In other embodiments, compounds of the invention are in the form of the (R) enantiomers.

In particular embodiments the compound of formula (1) is selected from the group consisting of:
N-[(3-cyclobutyl-2,3,4,5-tetrahydro-1H-3-benzazepin-7-yl)methyl]-2-[(3R)-3-methoxypyrrolidin-1-yl]pyrimidine-5-carboxamide
N-[(3-cyclobutyl-2,3,4,5-tetrahydro-1H-3-benzazepin-7-yl)methyl]-2-[(3S)-3-methoxypyrrolidin-1-yl]pyrimidine-5-carboxamide
N-((3-cyclobutyl-2,3,4,5-tetrahydro-1H-3-benzazepin-7-yl)methyl)-2-(3-methoxyazetidin-1-yl)pyrimidine-5-carboxamide;
N-[(3-cyclobutyl-2,3,4,5-tetrahydro-1H-3-benzazepin-7-yl)methyl]-2-(3,3-difluoropyrrolidin-1-yl)pyrimidine-5-carboxamide;
N-((3-cyclobutyl-2,3,4,5-tetrahydro-1H-3-benzazepin-7-yl)methyl)-2-(4,4-difluoropiperidin-1-yl)pyrimidine-5-carboxamide;
N-[(3-cyclobutyl-2,3,4,5-tetrahydro-1H-3-benzazepin-7-yl)methyl]-2-[(3S)-3-methoxypyrrolidin-1-yl]-N-methylpyrimidine-5-carboxamide;
N-[(3-cyclobutyl-2,3,4,5-tetrahydro-1H-3-benzazepin-7-yl)methyl]-2-[(3R)-3-methoxypyrrolidin-1-yl]-N-methylpyrimidine-5-carboxamide;
N-[(3-cyclobutyl-2,3,4,5-tetrahydro-1H-3-benzazepin-7-yl)methyl]-2-{[(2S)-2-methoxypropyl](methyl)amino}pyrimidine-5-carboxamide;
N-[(3-cyclobutyl-2,3,4,5-tetrahydro-1H-3-benzazepin-7-yl)methyl]-2-{[(2R)-2-methoxypropyl](methyl)amino}pyrimidine-5-carboxamide.

Particularly useful compounds in accordance with the invention include each of the compounds described in the accompanying examples, and pharmaceutically acceptable salts thereof.

In accordance with a second aspect of the invention, there is provided a pharmaceutical composition comprising a compound according to the first aspect of the invention, together with one or more pharmaceutically acceptable excipients.

Pharmaceutical compositions of this invention comprise any of the compounds of the first aspect of the present invention, or pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention are those conventionally employed in the field of pharmaceutical formulation, and include, but are not limited to, sugars, sugar alcohols, starches, ion exchangers, alumina, aluminium stearate, lecithin, serum proteins, such as human serum albumin, buffer substances such as phosphates, glycerine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, rectally, nasally, buccally, vaginally or via an implanted reservoir. Oral administration is preferred. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intraarticular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as that described in Ph. Helv, or a similar alcohol.

The pharmaceutical compositions of this invention may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

The compounds of the present invention may be administered in a dose of around 1 to around 20,000 μg/kg per dose, depending on the condition to be treated or prevented, and the characteristics of the subject being administered with the compound. In many instances, the dose may be around 1 to around 1500 μg/kg per dose. The dosing regimen for a given compound could readily be determined by the skilled person having access to this disclosure.

In one particular embodiment, the pharmaceutical composition of the invention additionally comprises one or more additional active pharmaceutical ingredients. These additional active ingredients may be agents known to the skilled person to be useful in the treatment or prevention of the diseases mentioned in the present disclosure.

In a third aspect, the present invention provides a compound according to the first aspect of the invention, or a composition according to the second aspect, for use in therapy.

In a fourth aspect, the invention provides a compound according to the first aspect of the invention, or a composition according to the second aspect, for use in the treatment or prevention of a condition whose development or symptoms are linked to histamine H3 receptor activity.

A number of conditions whose development or symptoms are linked to histamine H3 receptor activity are known to the skilled person.

In a fifth aspect, the invention also provides a method of treatment or prevention of a condition whose development or symptoms are linked to histamine H3 receptor activity, the method comprising the administration, to a subject in need of such treatment or prevention, of a therapeutically effective amount of a compound according to the first aspect of the invention, or a composition according to the second aspect.

A compound according to the fourth aspect, or a method according to the fifth aspect, wherein the condition is a disorder of the central nervous system.

In certain embodiments, the condition to be treated may be selected from sleep disorders (such as narcolepsy and hypersomnia), cognitive disorders (such as dementia and schizophrenia), attentional disorders (such as attention deficit hyperactivity disorder), neurodegenerative disorders (such as AD), schizophrenia, epilepsy, pain (such as neuropathic pain) and obesity.

In preferred embodiments the condition may be selected from schizophrenia, Alzheimer's Disease (AD) and dementia. In an alternative embodiment, the condition may be selected from narcolepsy, pain and obesity.

In particular embodiments, the condition may be selected from narcolepsy, neuropathic pain and obesity.

In a sixth aspect, the present invention provides the use of a compound according to the first aspect of the invention in the preparation of a medicament for the treatment or prevention of a condition whose development or symptoms are linked to histamine H3 receptor activity. Such conditions may be selected from those described above.

In a seventh aspect, the present invention provides a method for preparing a compound according to the first aspect of the invention. Preferably, the method of preparing the compound comprises the step of reacting an intermediate having the formula:

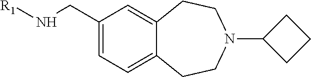

wherein R₁ is H or C₁₋₆ alkyl,
with a pyrimidine derivative of the formula:

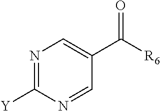

wherein Y represents —NR₂R₃ as depicted in formula (B), or a ring of formula (A)

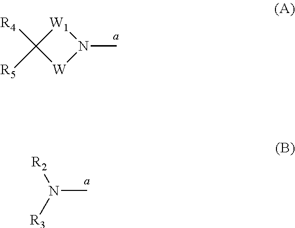

wherein ^{a} represents the point of attachment to the pyrimidinyl ring;
R₂ represents C₁₋₄alkyl substituted by C₁₋₃ alkoxy;
R₃ represents C₁₋₄ alkyl;
W represents —(CH₂)ₙ—;
W₁ represents —(CH₂)ₚ—;
n represents 1 or 2 or 3;
p represents 1 or 2;
R₄ represents C₁₋₄ alkoxy, C₁₋₆ alkyl or halogen; and
R₅ represents halogen or H,
provided that, when R₄ represents halogen, R₅ is not H; and
R₆ is OH or a carbonyl activating group.

The term ‘carbonyl activating group’ is intended to refer to groups that may be employed in order to activate a carbonyl group, thereby allowing the pyrimidine derivative to react with the other intermediate to form an amide bond. In effect, the —COR₆ group of the pyrimidine derivative can be a carboxylic acid group or a reactive derivative of a carboxylic acid. Such activating groups are well known to those skilled in the art. Examples include halides e.g. chloride or activated carboxylic acid derivatives that may be prepared in situ e.g. using N1-((ethylimino)methylene)-N3,N3-dimethylpropane-1,3-diamine hydrochloride and 3H-[1,2,3]triazolo[4,5-b]pyridin-3-ol, in a suitable solvent e.g. DMF. In one embodiment, R₆ is OH or a halide such as chloride.

Preferably, R₆ is OH or OR₇, where R₇ is a carboxyl activating group.

The term ‘carboxyl activating group’ is intended to refer to groups that may be employed in order to activate a carboxylic acid group, thereby allowing the pyrimidine derivative to react with the other intermediate to form an amide bond. Such groups are well known to those skilled in the art.

The carboxylic acid may be used as either the free acid or as a suitable salt e.g. Li. Typically the carboxylic acid is activated in situ e.g. using N1-((ethylimino)methylene)-N3,N3-dimethylpropane-1,3-diamine hydrochloride and 3H-[1,2,3]triazolo[4,5-b]pyridin-3-ol, in a suitable solvent e.g. DMF. The reaction mixture may then be carefully added to a solution of the amine in a suitable solvent e.g. THF and water in the presence of a base e.g. NaOH.

Novel intermediates form a further aspect of the invention.

In certain embodiments, the pyrimidine derivative represents a compound of formula (i):

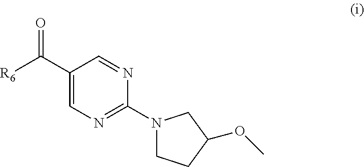

wherein R₆ is as herein defined.

In certain other embodiments, the pyrimidine derivative represents a compound of formula (ii):

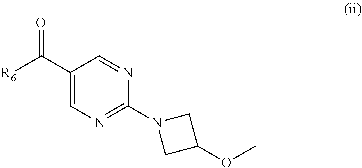

wherein R₆ is as herein defined.

The invention will now be described in more detail by way of example only.

### 1. SYNTHETIC METHODOLOGIES

The methods used for synthesis of the compounds of the invention are illustrated by the general scheme below and the preparative examples that follow. All compounds and intermediates were characterised at least by liquid chromatography-mass spectroscopy (LCMS). The starting materials and reagents used in preparing these compounds are available from commercial suppliers. These general schemes are merely illustrative of methods by which the compounds of this invention can be synthesised, and various modifications to these schemes can be made and will be suggested to one skilled in the art having referred to this disclosure.

Nuclear magnetic resonance (NMR) spectra were recorded at 400 MHz unless otherwise stated; the chemical shifts (δ) are reported in parts per million.

Mass spectra were recorded using an LCMS system (ZQ mass spec detector).

Compounds were purified using normal phase chromatography on silica or alumina, or by reverse phase chromatographic methods.

Room temperature in the following schemes means the temperature ranging from 20° C. to 25° C.

The desired compounds of Formula 1 can be prepared as outlined in Schemes 1 and 3, or Schemes 1, 2 and 3, as follows:

### LIST OF ABBREVIATIONS

### Ac Acetyl

### AcOH Acetic Acid

### Aq Aqueous

### Boc Tert-butoxycarbonyl

(Boc)₂O Di-tert-butyl dicarbonate

### DCM Dichloromethane

### DIPEA Diisopropylethylamine

### DMSO Dimethyl Sulfoxide

### DMF Dimethyl Formamide

### Et Ethyl

### EtOAc Ethyl Acetate

### EtOH Ethanol

### Et₃N Triethylamine

### IPE Di-isopropyl Ether

### LCMS Liquid Chromatography Mass Spectrum

### MS Mass Spectrum

### MeOD Deuterated Methanol

### MeOH Methanol

### MeONH₂ Methoxyamine

MTBE Methyl tert-butyl ether

### Min Minute

NaBH(OAc)₃ Sodium triacetoxyborohydride

### NMR Nuclear Magnetic Resonance

### Ph Phenyl

### RT Room Temperature

Sat. Saturated

### THF Tetrahydrofuran

### TLC Thin Layer Chromatography

In the following schemes, R₁, R₂, R₃, R₄, R₅, R₆ and Y are as defined above.

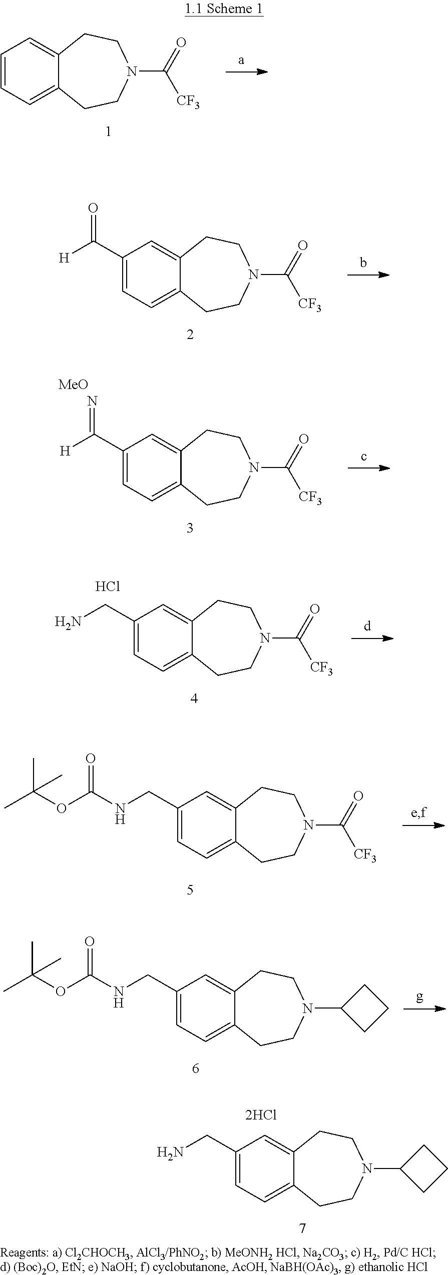

### 1.1.1 Intermediate 1

The benzazepine intermediate (1) can be prepared by methods outlined in WO 2005/058328 and WO 2005/094834.

### 1.1.2 Intermediate 2

To a mixture of 3-(trifluoroacetyl)-2,3,4,5-tetrahydro-1H-3-benzazepine (24.3 g, 0.10 mol) (1) and PhNO₂ (24 mL), was added AlCl₃ (26.7 g, 0.20 mol) at 5° C. (internal temperature) in one portion and stirred for 15 min. To the resulting mixture, was added a solution of Cl₂CHOCH₃ (34.5 g, 0.30 mol) in PhNO₂ (24 mL) dropwise at 5° C. over 50 min and the mixture was stirred at room temperature for 8 h. The reaction mixture was diluted with EtOAc (100 mL) and poured onto ice (150 g) carefully. The mixture was extracted with EtOAc (100 mL×2) and was washed with water (50 mL×2). The combined organic layers were washed with brine (200 mL), dried over MgSO₄ and concentrated. The residue was purified by column chromatography on SiO₂ (350 g) (EtOAc/hexane=1/20˜3/7) to give crude solid (25.0 g). The obtained solid was dissolved in IPE (30 mL) and hexane (90 mL) was added dropwise to the solution with stirring at 50° C. The mixture was cooled to room temperature and was stirred for 30 min. The deposited precipitate was filtered and was washed (EtOAc/hexane=1/5, 50 mL) to give 3-(trifluoroacetyl)-2,3,4,5-tetrahydro-1H-3-benzazepine-7-carbaldehyde as pale yellow powder (20.3 g, 74.8%).

¹H-NMR (300 MHz, CDCl₃) δ: 3.05-3.10 (4H, m), 3.72-3.82 (4H, m), 7.31-7.72 (2H, m), 9.981 (1H, s). MS (ES⁺) 272

### 1.1.3 Intermediate 3

To a solution of Na₂CO₃ (6.36 g, 0.06 mol) in water (140 mL), was added MeONH₂HCl (10.0 g, 0.12 mol) portion wise at 5° C. (internal temperature) and stirred for 30 min. To the mixture, was added a solution of 3-(trifluoroacetyl)-2,3,4,5-tetrahydro-1H-3-benzazepine-7-carbaldehyde (27.1 g, 0.1 mol) in THF (140 mL) drop wise at 5° C. and the mixture was stirred at room temperature for 2 h. The reaction mixture was diluted with EtOAc (280 mL) and undissolved material was filtered. The separated aqueous layer was extracted with EtOAc (140 mL) and organic layers were combined and washed with brine (140 mL), and then dried over MgSO₄. The solvent was evaporated under reduced pressure to afford yellow oil (31 g) which was dissolved in IPE (62 mL) and then hexane (124 mL) was added drop wise with stirring. The precipitate appeared was collected by filtration and washed with IPE:hexane (1:2, 50 mL), and then was dried under reduced pressure to give 3-(trifluoroacetyl)-2,3,4,5-tetrahydro-1H-3-benzazepine-7-carbaldehyde O-methyloxime as pale yellow powder (23.0 g, 76.6%).

¹H-NMR (400 MHz, CDCl₃) δ: 2.97-3.02 (4H, m), 3.68-3.71 (2H, m), 3.76-3.78 (2H, m), 3.97 (3H, s), 7.13-7.18 (1H, m), 7.33-7.36 (1H, m), 7.41-7.44 (1H, m), 8.03 (1H, s). MS (ES⁺) 301

### 1.1.4 Intermediate 4

To a solution of 3-(trifluoroacetyl)-2,3,4,5-tetrahydro-1H-3-benzazepine-7-carbaldehyde O-methyloxime (21 g, 0.07 mol) in MeOH (420 mL) and aqueous 12 M HCl (5.3 mL, 175 mmol), was added 10% Pd/C (wet 50%, 2.1 g) and the mixture was hydrogenated under an atmospheric pressure at room temperature for 1 h. The catalyst was removed by filtration and the filtrate was concentrated under reduced pressure. The resulting solid was treated with IPE (200 mL) and was collected by filtration, and then was dried under reduced pressure to give 1-[3-(trifluoroacetyl)-2,3,4,5-tetrahydro-1H-3-benzazepin-7-yl]methanamine hydrochloride (20.1 g, 92.8%) as white solid.

¹H-NMR (400 MHz, DMSO-d₆) δ: 2.96-3.02 (4H, m), 3.66-3.71 (4H, m), 3.96 (2H, s), 7.21-7.30 (3H, m), 8.33 (3H, broad s).

MS (ES⁺) 273

### 1.1.5 Intermediate 5

To a solution of 1-[3-(trifluoro acetyl)-2,3,4,5-tetrahydro-1H-3-benzazepin-7-yl]methanamine hydrochloride (18.5 g, 60 mmol) in THF (90 mL) and water (82 mL), was added (Boc)₂O (13.1 g, 60 mmol) in one portion at 5° C. (internal temperature), and then aqueous 8M NaOH (7.5 mL, 60 mL) solution dropwise at the same temperature. The mixture was stirred at room temperature for 1 h. The reaction mixture was extracted with EtOAc (90 mL×2) and combined organic layers were washed with brine (90 mL), and then dried over MgSO₄. The solvent was evaporated under reduced pressure to give light brown syrup, which was treated with hexane (70 mL) to afford white precipitate. The obtained precipitate was collected by filtration and washed with hexane (20 mL), and then was dried under reduced pressure to give) tent-butyl {[3-(trifluoroacetyl)-2,3,4,5-tetrahydro-1H-3-benzazepin-7-yl]methyl}carbamate (21.0 g, 94%) as white powder.

¹H-NMR (400 MHz, CDCl₃) δ: 1.46 (9H, s), 2.94-2.99 (4H, m), 3.67-3.69 (2H, m), 3.74-3.78 (2H, m), 4.27-4.29 (2H, m), 4.83 (1H, broad s), 7.06-7.14 (3H, m).

### 1.1.6 Intermediate 6

To a solution of tert-butyl {[3-(trifluoroacetyl)-2,3,4,5-tetrahydro-1H-3-benzazepin-7-yl]methyl}carbamate (16.8 g, 45.0 mmol) in MeOH (170 mL), was added aqueous 8 M NaOH solution (6.2 mL, 49.5 mmol) at 5° C. (internal temperature) and the mixture was stirred at room temperature for 1 h. To the resulting mixture, were added AcOH (3.9 mL, 67.5 mmol), cyclobutanone (4.7 g, 67.5 mmol), and NaBH(OAc)₃ (14.3 g, 67.5 mmol) at 5° C. and the mixture was stirred at room temperature for 3 h. To the mixture, were added cyclobutanone (4.7 g, 67.5 mmol) and NaBH(OAc)₃ (14.3 g, 67.5 mmol) again and the mixture was stirred at room temperature for 1 h. The reaction mixture was concentrated under reduced pressure and the residue was treated with water (150 mL). The aqueous mixture was made basic (pH=9) with aqueous NaOH solution under cooling and was extracted with EtOAc (150 mL×2). The combined organic layers were washed with brine (150 mL) and dried over MgSO₄. The solution was subjected to short silica-gel pad (40 g) and the solvent was evaporated under reduced pressure. The obtained solid was treated with hexane:IPE (1:1, 100 mL) and was collected by filtration. The solid was washed with hexane (10 mL) and was dried under reduced pressure to give tert-butyl [(3-cyclobutyl-2,3,4,5-tetrahydro-1H-3-benzazepin-7-yl)methyl]carbamate (12.4 g, 83.3%) as a white solid.

¹H-NMR (400 MHz, CDCl₃) δ: 1.55-1.75 (2H, m), 1.85-1.97 (2H, m), 2.03-2.12 (2H, m), 2.35-2.50 (4H, m), 2.72-2.81 (1H, m), 2.87-2.94 (4H, m), 4.25-4.27 (2H, m), 4.78 (1H, s), 7.01-7.07 (3H, m). MS (ES⁺) 331

### 1.1.7 Intermediate 7

A mixture of tert-butyl [(3-cyclobutyl-2,3,4,5-tetrahydro-1H-3-benzazepin-7-yl)methyl]carbamate (11.6 g, 35.0 mmol) and 2M ethanolic HCl solution (87.5 mL, 175 mmol) was warmed at 50° C. for 30 min. The reaction mixture was cooled in an iced water bath and treated with IPE (100 mL). The deposited precipitate was collected by filtration and was washed with IPE (20 mL), and then was dried under reduced pressure to give 1-(3-cyclobutyl-2,3,4,5-tetrahydro-1H-3-benzazepin-7-yl)methanamine dihydrochloride (9.5 g, 90%) as white powder.

¹H-NMR (400 MHz, DMSO-d₆) δ: 1.58-1.74 (2H, m), 2.15-2.17 (2H, m), 2.49-2.54 (2H, m), 2.68-2.71 (2H, m), 2.94-3.00 (2H, m), 3.50-3.52 (4H, m), 3.64-3.66 (1H, m), 7.23-7.26 (1H, m), 7.33-7.34 (2H, m), 8.56 (3H, s), 11.94 (1H, s) MS (ES⁺) 231

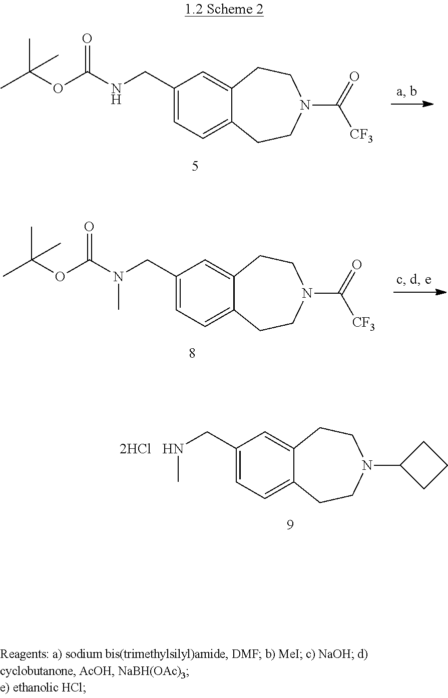

### 1.2.1 Intermediate 8

A round-bottomed flask was charged with tert-butyl {[3-(trifluoroacetyl)-2,3,4,5-tetrahydro-1H-3-benzazepin-7-yl]methyl}carbamate (3.7 g, 10 mmol) (Intermediate 5) in DMF (10 mL), the reaction was cooled in an ice bath and sodium bis(trimethylsilylamide) (2.73 g, 15 mmol in THF, 14.9 mL) was added dropwise to give a yellow solution. The reaction was stirred for an hour whilst maintaining cooling and iodomethane (0.93 ml, 15 mmol) was added. The reaction was stirred for 16 hours and then the reaction mixture was diluted with ethyl acetate and washed with brine (×5). The organic layer was dried and evaporated and the residue was purified by column chromatography on silica using (10-100%) ethyl acetate in petrol to yield tert-butyl methyl{[3-(trifluoroacetyl)-2,3,4,5-tetrahydro-1H-3-benzazepin-7-yl]methyl}carbamate (3.2 g, 83% yield) as a white solid.

¹H NMR (400 MHz, CD₂Cl₂) δ 6.99-7.08 (m, 1H), 6.89-6.99 (m, 2H), 4.27 (s, 2H), 3.62-3.70 (m, 2H), 3.54-3.62 (m, 2H), 2.83-2.95 (m, 4H), 2.70 (s, 3H), 1.37 (s, 9H)

MS (ES⁺) 287 (M+H-Boc)

### 1.2.2 Intermediate 9

A round-bottomed flask was charged with tent-butyl methyl {[3-(trifluoroacetyl)-2,3,4,5-tetrahydro-1H-3-benzazepin-7-yl]methyl}carbamate (1.7 g, 4.40 mmol) and sodium hydroxide (0.6 mL (2M, aq.), 4.80 mmol) in methanol (20 mL) to give a colourless solution. The reaction was stirred for 16 hours and then acetic acid (0.68 mL, 12 mmol) and cyclobutanone (0.21 g, 12 mmol) and sodium triacetoxyborohydride, (2.6 g, 12 mmol) were added. The reaction was stirred for 16 hours. The reaction was diluted with ethyl acetate and washed with sodium hydroxide. The organic layer was dried and evaporated and the residue was purified by column chromatography on silica using 0-20% methanol in dichloromethane on silica (with ammonia). The residue was treated with ethanolic hydrochloric acid to yield 1-(3-cyclobutyl-2,3,4,5-tetrahydro-1H-3-benzazepin-7-yl)-N-methylmethanamine dihydrochloride (0.9 g, 75%).

¹H NMR (400 MHz, CD₃OD) δ 7.30-7.44 (m, 3H), 4.19 (s, 2H), 3.63-3.82 (m, 3H), 3.35-3.50 (m, 2H), 3.09-3.22 (m, 2H), 2.78-2.91 (m, 2H), 2.73 (s, 3H), 2.31-2.55 (m, 4H), 1.71-2.03 (m, 2H)

MS ES⁺ 281

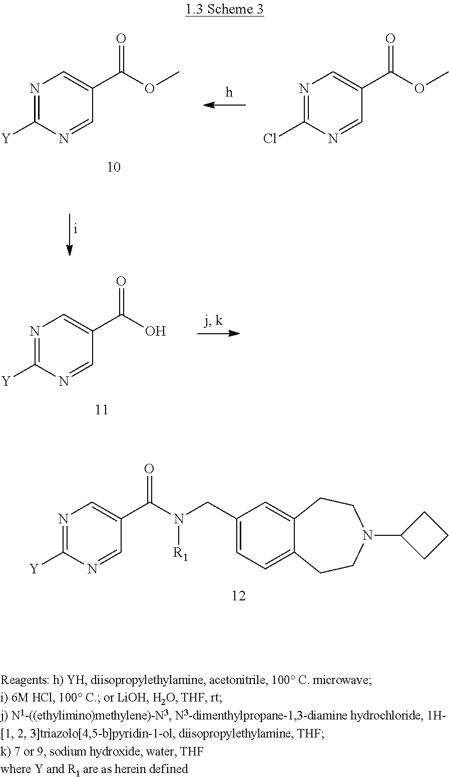

It will be appreciated that the carboxylic acid (11) may be used as either the free acid or as a suitable salt e.g. Li.

1.3.1

### Intermediate 10A

Diisopropylethylamine (1.5 mL, 8.7 mmol) was added to a mixture of methyl 2-chloropyrimidine-5-carboxylate (0.5 g, 2.9 mmol) and (3R)-3-methoxypyrrolidine hydrochloride (0.48 g, 3.48 mmol) in acetonitrile (6 mL) and the mixture was microwaved at 140° C. for 30 min. The reaction was diluted with ethyl acetate (20 mL), water (10 mL) and sodium carbonate (sat., aq., 10 mL). The phases were separated and the aqueous phase was re-extracted with ethyl acetate (2×20 mL) and the combined organic phases were washed with water (10 mL), brine (2×10 mL), dried (MgSO₄), filtered and concentrated. The residue was purified by silica chromatography, 0-100% ethyl acetate in petrol to give product as a light yellow solid methyl 2-[(3R)-3-methoxypyrrolidin-1-yl]pyrimidine-5-carboxylate (0.66 g, 95% yield).

¹H NMR (400 MHz, CD₃OD) δ 8.80 (s, 2H), 4.10-4.16 (m, 1H), 3.86 (s, 3H), 3.71-3.81 (m, 2H), 3.55-3.70 (m, 2H), 3.36 (s, 3H), 2.04-2.24 (m, 2H)

MS (ES⁺) 238

### Intermediate 10B

A mixture of methyl 2-chloropyrimidine-5-carboxylate (0.75 g, 4.35 mmol), 3-methoxyazetidine hydrochloride (0.81 g, 6.5 mmol) and DIPEA (2.27 mL, 13.0 mmol) in acetonitrile (5 mL) was microwaved at 140° C. for 30 min. The reaction was then diluted with EtOAc (40 mL) and Na₂CO₃ (sat. aq., 15 mL). The phases were separated and the aqueous phase extracted with EtOAc (2×15 mL). The combined organic phases were washed with brine (15 mL), dried (MgSO₄), filtered and concentrated. Purified by Biotage Si column, 20-100% EtOAc/petrol to give methyl 2-(3-methoxyazetidin-1-yl)pyrimidine-5-carboxylate (908 mg, 94%).

¹H NMR (400 MHz, CD₃OD) δ ppm 8.78 (s, 2H) 4.28-4.52 (m, 3H) 3.96-4.13 (m, 2H) 3.89 (s, 3H) 3.37 (s, 3H).

MS (ES⁺) 224

1.3.2

### Intermediate 11A

A solution of methyl 2-[(3R)-3-methoxypyrrolidin-1-yl]pyrimidine-5-carboxylate (0.65 g, 2.7 mmol) in HCl (18% aq.) (15 mL, 77 mmol) was heated to 95° C. overnight and then cooled and concentrated. The residue was azeotroped with toluene (×3) and then dried in vacuum oven over phosphorous pentoxide to yield a white solid 2-[(3R)-3-methoxypyrrolidin-1-yl]pyrimidine-5-carboxylic acid hydrochloride (646 mg, 91% yield).

¹H NMR (400 MHz, DMSO-d₆) δ 8.70-8.82 (m, 2H), 4.04-4.15 (m, 1H), 3.45-3.74 (m, 4H), 3.27 (s, 3H), 1.99-2.15 (m, 2H)

MS (ES⁺) 224

### Intermediate 11B

To a solution of methyl 2-(3-methoxyazetidin-1-yl)pyrimidine-5-carboxylate (12 g, 53.8 mmol) in THF (90 ml) and water (90 ml) was added LiOH (1.545 g, 64.5 mmol) and the reaction stirred for 18 h. The mixture was acidified to pH1 via the addition of 1N HCl (aq) and then extracted with EtOAc. The organic extracts were dried (MgSO₄) and concentrated under reduced pressure to give 2-(3-methoxyazetidin-1-yl)pyrimidine-5-carboxylic acid (6.8 g, 61%).

¹H NMR (400 MHz, DMSO-d₆) δ ppm 8.75 (s, 2H) 4.28-4.39 (m, 3H) 3.81-4.00 (m, 2H) 3.27 (s, 3H)

MS (ES⁺) 210

Alternatively the lithium salt is isolated as below;

To a solution of methyl 2-(3-methoxyazetidin-1-yl)pyrimidine-5-carboxylate (0.806 g, 3.61 mmol) in THF (15 mL) and water (10 mL) was added lithium hydroxide (0.104 g, 4.33 mmol) and the mixture was stirred at RT for 5 h. Concentrated and azeotroped with toluene and then dried over P₂O₅ in a vacuum oven for 3 days.

1.3.3

### Compound 12A (Formula 1-Ex 1)

To a suspension of 2-[(3R)-3-methoxypyrrolidin-1-yl]pyrimidine-5-carboxylic acid hydrochloride (0.65 g, 2.5 mmol) in DMF (3 mL) was added diisopropylethylamine (0.87 mL, 5 mmol), resulting in dissolution of the solid. 3H-[1,2,3]triazolo[4,5-b]pyridin-3-ol (0.41 g, 3 mmol) and N1-((ethylimino)methylene)-N3,N3-dimethylpropane-1,3-diamine hydrochloride (0.6 g, 3 mmol) were added and the mixture was stirred for 1 hour before adding at 0° C. the activated ester, with a DMF (1 mL) rinse to a solution of (3-cyclobutyl-2,3,4,5-tetrahydro-1H-3-benzazepin-7-yl)methanamine dihydrochloride (0.69 g, 2.3 mmol) in THF (1 mL) and water (1 mL). 2.1 mL of 2M sodium hydroxide was added. The mixture was stirred for 2.5 hours and then sodium bicarbonate (sat., aq., 10 mL) and water (10 mL) were added and the mixture was extracted with ethyl acetate (3×20 mL). Combined organic phases washed with water (2×10 mL), brine (3×10 mL), dried (MgSO4), filtered and concentrated. Isopropyl ether (15 mL) added and stirred for 30 min, heptane (7 mL) added and stirred for 1 h. White solid collected by filtration N-[(3-cyclobutyl-2,3,4,5-tetrahydro-1H-3-benzazepin-7-yl)methyl]-2-[(3R)-3-methoxypyrrolidin-1-yl]pyrimidine-5-carboxamide (647 mg, 65.7% yield)

¹H NMR (400 MHz, CD₃OD) δ 8.77 (s, 2H), 7.04-7.11 (m, 3H), 4.48 (s, 2H), 4.10-4.15 (m, 1 H), 3.52-3.80 (m, 4H), 3.36 (s, 3H), 2.88-2.95 (m, 4H), 2.78-2.88 (m, 1H), 2.46 (s, 4H), 2.15-2.25 (m, 1H), 2.03-2.15 (m, 3H), 1.86-2.00 (m, 2H), 1.59-1.77 (m, 2H).

MS ES+ 436

### Compound 12B (Formula 1-Ex 3)

To a solution of 2-(3-methoxyazetidin-1-yl)pyrimidine-5-carboxylic acid (6.8 g, 32.5 mmol) in DMF (70 ml), EDC (7.48 g, 39.0 mmol) and 1-hydroxy-7-azabenzotriazole (5.31 g, 39.0 mmol) were added. Separately (3-cyclobutyl-2,3,4,5-tetrahydro-1H-3-benzazepin-7-yl)methanamine (9.86 g, 32.5 mmol) was taken up in THF (17.50 ml) and water (17.50 ml) and NaOH (aq) (65.0 ml, 65.0 mmol) was added to give a thick slurry. Both mixtures were stirred at RT for 1 h. The solution of the activated ester was then added to the amine and the resultant mixture stirred for 1 h. Saturated aq. NaHCO₃ and EtOAc were added. THF was also added to aid solution. The layers were separated and the org phase washed with brine, dried (MgSO₄) and concentrated under reduced pressure. Purified by column chromatography (SiO₂, DCM to 15% MeOH(NH₃)), followed by trituration from MTBE and recrystallisation from EtOH to give N-((3-cyclobutyl-2,3,4,5-tetrahydro-1H-3-benzazepin-7-yl)methyl)-2-(3-methoxyazetidin-1-yl)pyrimidine-5-carboxamide (6.9 g, 50%)

Example compounds of the inventions were synthesized according to Scheme 3 unless otherwise indicated.

### 2. EXAMPLE COMPOUNDS

### 2.1 Example 1

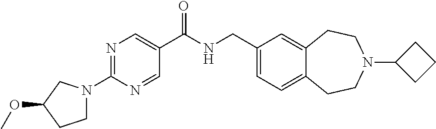 [0127] N-[(3-cyclobutyl-2,3,4,5-tetrahydro-1H-3-benzazepin-7-yl)methyl]-2-[(3R)-3-methoxypyrrolidin-1-yl]pyrimidine-5-carboxamide

Prepared according to Scheme 3 where YH is (3R)-3-methoxypyrrolidine and using Intermediate 7

¹H NMR (400 MHz, CD₃OD) δ 8.77 (s, 2H), 7.04-7.11 (m, 3H), 4.48 (s, 2H), 4.10-4.15 (m, 1 H), 3.52-3.80 (m, 4H), 3.36 (s, 3H), 2.88-2.95 (m, 4H), 2.78-2.88 (m, 1H), 2.46 (br. s., 4H), 2.15-2.25 (m, 1H), 2.03-2.15 (m, 3H), 1.86-2.00 (m, 2H), 1.59-1.77 (m, 2H).

MS ES⁺ 436

### 2.2 Example 2

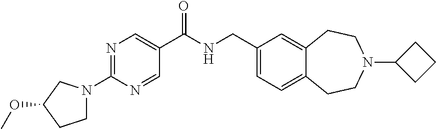 [0132] N-[(3-cyclobutyl-2,3,4,5-tetrahydro-1H-3-benzazepin-7-yl)methyl]-2-[(3S)-3-methoxypyrrolidin-1-yl]pyrimidine-5-carboxamide

Prepared according to Scheme 3 where YH is (3S)-3-methoxypyrrolidine and using Intermediate 7

¹H NMR (400 MHz, CD₃OD) δ 8.77 (s, 2H), 7.04-7.11 (m, 3H), 4.48 (s, 2H), 4.10-4.15 (m, 1 H), 3.52-3.80 (m, 4H), 3.36 (s, 3H), 2.88-2.95 (m, 4H), 2.78-2.88 (m, 1H), 2.46 (s, 4H), 2.15-2.25 (m, 1H), 2.03-2.15 (m, 3H), 1.86-2.00 (m, 2H), 1.59-1.77 (m, 2H).

MS ES⁺ 436

### 2.3 Example 3

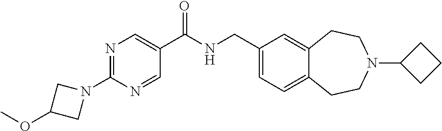 [0137] N-((3-cyclobutyl-2,3,4,5-tetrahydro-1H-3-benzazepin-7-yl)methyl)-2-(3-methoxyazetidin-1-yl)pyrimidine-5-carboxamide

Prepared according to Scheme 3 where YH is 3-methoxyazetidine and using Intermediate 7

¹H NMR (400 MHz, CD₃OD) δ 8.76 (s, 2H), 7.03-7.13 (m, 3H), 4.48 (s, 2H), 4.31-4.40 (m, 3 H), 3.93-4.07 (m, 2H), 3.35 (s, 3H), 2.87-2.95 (m, 4H), 2.77-2.87 (m, 1H), 2.46 (s, 4H), 2.04-2.15 (m, 2H), 1.86-2.01 (m, 2H), 1.60-1.78 (m, 2H).

MS ES⁺ 422

### 2.4 Example 4

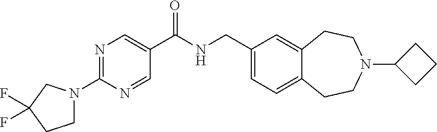 [0142] N-[(3-cyclobutyl-2,3,4,5-tetrahydro-1H-3-benzazepin-7-yl)methyl]-2-(3,3-difluoropyrrolidin-1-yl)pyrimidine-5-carboxamide

Prepared according to Scheme 3 where YH is 3,3-difluoropyrrolidine and using Intermediate 7

¹H NMR (400 MHz, D₆-DMSO) δ 8.80-8.93 (m, 3H), 6.99-7.09 (m, 3H), δ 4.33-4.46 (m, 2H), 3.90-4.04 (m, 2H), 3.70-3.85 (m, 2H), 2.69-2.87 (m, 5H), 2.50-2.63 (m, 2H), 2.26-2.44 (m, 4H), 1.94-2.06 (m, 2H), 1.71-1.86 (m, 2H), 1.48-1.68 (m, 2H)

MS ES⁺ 442

### 2.5 Example 5

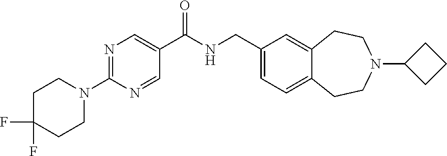 [0147] N-((3-cyclobutyl-2,3,4,5-tetrahydro-1H-3-benzazepin-7-yl)methyl)-2-(4,4-difluoropiperidin-1-yl)pyrimidine-5-carboxamide

Prepared according to Scheme 3 where YH is 4,4-difluoropiperidine and using Intermediate 7

¹H NMR (400 MHz, DMSO-d₆) δ 8.85 (s, 3H), 6.95-7.10 (m, 3H), 4.33-4.45 (m, 2H), 3.85-4.07 (m, 4H), 2.70-2.92 (m, 5H), 2.21-2.39 (m, 4H), 1.89-2.11 (m, 6H), 1.68-1.86 (m, 2H), 1.42-1.69 (m, 2H)

MS ES⁺ 456

### 2.6 Example 6

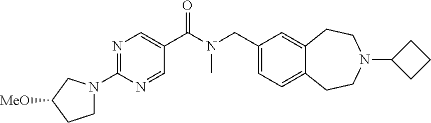 [0152] N-[(3-cyclobutyl-2,3,4,5-tetrahydro-1H-3-benzazepin-7-yl)methyl]-2-[(3S)-3-methoxypyrrolidin-1-yl]-N-methylpyrimidine-5-carboxamide

Prepared according to Scheme 3 where YH is (3S)-3-methoxypyrrolidine and using Intermediate 9

¹H NMR (400 MHz, CD₃OD) δ 8.47 (s. 2H), 6.97-7.16 (m, 3H), 4.64 (s, 2H), 4.08-4.15 (m, 1H), 3.66-3.77 (m, 2H), 3.52-3.65 (m, 2H), 3.35 (s, 3H), 3.02 (s, 3H), 2.93 (s, 4H), 2.78-2.89 (m, 1H), 2.47 (br. s., 4H), 2.02-2.23 (m, 4H), 1.87-2.01 (m, 2H), 1.60-1.77 (m, 2H).

MS ES⁺ 450

### 2.7 Example 7

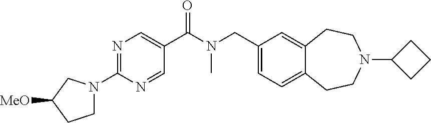 [0157] N-[(3-cyclobutyl-2,3,4,5-tetrahydro-1H-3-benzazepin-7-yl)methyl]-2-[(3R)-3-methoxypyrrolidin-1-yl]-N-methylpyrimidine-5-carboxamide

Prepared according to Scheme 3 where YH is (3R)-3-methoxypyrrolidine and using Intermediate 9

¹H NMR (400 MHz, CD₃OD) δ 8.47 (s, 2H), 7.08-7.17 (m, 1H), 7.04 (s, 2H), 4.64 (s, 2H), 4.07-4.16 (m, 1H), 3.67-3.81 (m, 2H), 3.51-3.67 (m, 2H), 3.36 (s, 3H), 3.03 (s, 3H), 2.89-2.98 (m, 4H), 2.79-2.89 (m, 1H), 2.49 (s, 4H), 2.01-2.25 (m, 4H), 1.87-2.02 (m, 2H), 1.59-1.79 (m, 2H).

MS ES⁺: 450

### 2.8 Example 8

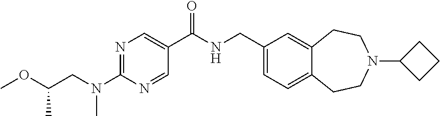 [0162] N-[(3-cyclobutyl-2,3,4,5-tetrahydro-1H-3-benzazepin-7-yl)methyl]-2-{[(2S)-2-methoxypropyl](methyl)amino}pyrimidine-5-carboxamide

Prepared according to Scheme 3 where YH is (2S)-2-methoxy-N-methylpropan-1-amine and using Intermediate 7

1H NMR (400 MHz, DMSO-d6) δ 8.65-8.92 (m, 3H), 6.89-7.18 (m, 3H), 4.28-4.52 (m, 2H), 3.55-3.77 (m, 3H), 3.23 (s, 3H), 3.19 (s, 3H), 2.66-2.89 (m, 5H), 2.26-2.42 (m, 4H), 1.90-2.09 (m, 2H), 1.69-1.88 (m, 2H), 1.40-1.68 (m, 2H), 0.91-1.18 (m, 3H)

MS (ES⁺) 438

### 2.9 Example 9

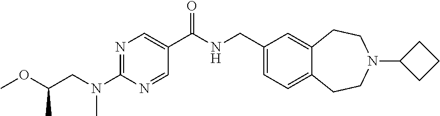 [0167] N-[(3-cyclobutyl-2,3,4,5-tetrahydro-1H-3-benzazepin-7-yl)methyl]-2-{[(2R)-2-methoxypropyl](methyl)amino}pyrimidine-5-carboxamide

Prepared according to Scheme 3 where YH is (2R)-2-methoxy-N-methylpropan-1-amine and using Intermediate 7

¹H NMR (400 MHz, DMSO-d₆) δ 8.79 (s, 3H), 6.96-7.10 (m, 3H), 4.32-4.47 (m, 2H), 3.55-3.75 (m, 3H), 3.23 (s, 3H), 3.19 (s, 3H), 2.68-2.86 (m, 5H), 2.33 (s, 4H), 1.92-2.05 (m, 2H), 1.69-1.85 (m, 2H), 1.49-1.66 (m, 2H), 1.01-1.10 (m, 3H).

MS (ES⁺) 438

### 3. BIOLOGICAL EFFICACY OF COMPOUNDS OF THE INVENTION

### 3.1 In Vitro H3 Binding Assay

The ability of compounds to bind to the H3 receptor was determined by measuring the reduction in tritiated N-α-methyl-histamine (³H-NαMH) binding in a competition binding assay. Changes in the levels of bound radio-label were monitored by scintillation counting with a Trilux Microbeta (Perkin Elmer).

Membranes were prepared from CHO-K1 cells stably expressing human H3 receptor; routinely grown as monolayers in Ham's F12 medium (Invitrogen) supplemented with 10% Foetal Clone III (Hyclone), 500 μg/ml G418 (Invitrogen), 5 μg/ml blasticidine S (Invivogen) and 50 μg/ml Gentamicin (Sigma) in 5% CO₂ at 37° C. Cells were grown to 80-95% confluency, rinsed once with 1×PBS (Invitrogen) and detached by incubating with 1×PBS containing 0.02% EDTA (Sigma) for 10 minutes at room temperature. Cells were collected by centrifugation at 900×g, 4° C. for 10 minutes. Cells were rinsed once with 1×PBS and re-suspended in ice cold homogenisation buffer (50 mM Tris-HCl (pH 7.4), 2.5 mM EDTA, 5 mM MgCl₂, 200 mM Sucrose) at 1×10⁷ cells/ml and kept on ice. Cells were homogenised on ice and debris removed by centrifugation at 500×g, 4° C. for 5 minutes. The resulting supernatant was centrifuged at 75,600×g, 4° C. for 60 minutes. Membranes were suspended in homogenisation buffer, protein concentration was determined (BCA Protein Assay kit (Pierce)), diluted to 2.2 mg/ml, dispensed into 1 ml aliquots and stored at −80° C.

Membranes were thawed on ice, sonicated with 4 cycles of 20 pulses (50% amplitude, 0.5 pulse) (UP200S Hielscher) on ice, diluted in assay buffer (50 mM Tris-HCl (pH7.4), 5 mM MgCl₂) to 62.5 μg/ml. Compound was serially diluted in DMSO before being diluted 1:10 with assay buffer. 5 μg of membrane in 80 μl of assay buffer was added per well of a 96 well polystyrene plate (Corning). 10 μl of compound was added per well. The assay was initiated by the addition of 10 μl of 20 nM ³H-NαMH per well and incubated for one hour at room temperature with shaking Total binding was determined in the presence of 1% DMSO and non-specific binding was determined by the inclusion of 1 μM R-α-methyl-histamine (RαMH). Incubations were then filtered through filtermat A (Perkin Elmer) and washed three times with assay buffer. Filtermats were dried at 42° C. for two hours, scintillant added and the level of bound radioactivity determined.

IC50 values for compounds were determined from seven point log scale dose-response studies and represent the concentration of compound required to inhibit 50% of the specific binding of 2 nM ³H-NαMH (difference between total and non-specific binding). Curves were generated using the average of duplicate wells for each data point and analyzed using nonlinear regression of sigmoidal dose response (variable slope).

### 3.2 In Vitro H3 Functional Assay

The functional activity of compounds at the H3 receptor was determined by measuring changes in the level of intracellular cAMP using a cAMP response element driven luciferase reporter assay. The changes in luciferase expression were monitored by a luminescence plate reader, Analyst HT (MDS Analytical). Increases in intracellular cAMP were readily detected upon activation of protein kinase A by forskolin (Sigma) and suppression of this response observed with the application of the H3 receptor agonist RαMH (Sigma).

CHO(dhfr⁺)-cre-luc cells stably expressing human H3 receptor were routinely grown as monolayers in Minimal Essential Medium α (MEMα) (Invitrogen) supplemented with 10% dialysed FBS (Hyclone), in 5% CO₂ at 37° C. 48 hours prior to assay, cells were seeded in clear-base white walled 384-well plates (Corning) at a density of 5000 cells/well. On the day of assay, growth media was removed and replaced with 15 μl of assay buffer (MEMα, 5 mg/ml fatty acid free BSA (Sigma)) per well. Cells were then incubated for 30 minutes at 37° C., 5% CO₂. Compound was serially diluted in DMSO before being diluted 1:10 with assay buffer. 2.5 μl of compound diluted in assay buffer was added and cells incubated for 5 minutes at 37° C., 5% CO₂. 2.5 μl of each reagent was then added in the following order: RαMH (10 nM), isobutylmethylxanthine (1-methyl-3-(2-methylpropyl)-7H-purine-2,6-dione; IBMX) (500 μM) (Sigma) and forskolin (1 μM). Cells were then incubated for 90 minutes at 37° C., 5% CO₂, followed by 30 minutes at room temperature. At the end of incubation 25 μl of Steadylite reagent (Perkin Elmer) was added, plates were sealed and placed on a shaker for 5 minutes. The level of light output to determine the level of luciferase expression was then measured.

IC50 values for compounds were determined from ten point half log scale dose-response studies and represent the concentration of compound required to prevent 50% inhibition of forskolin stimulated cells in the presence of RαMH alone. Curves were generated using the average of duplicate wells for each data point and analyzed using nonlinear regression of four parameter dose response.

### 3.3 Results

| Example | | |
|---|---|---|
| Compound | hH3 binding IC₅₀/nM | hH3 functional IC₅₀/nM |
| Example 1 | 0.5 | 1 |
| Example 2 | 2.0 | 2 |
| Example 3 | 3.0 | 2 |
| Example 4 | 2.0 | 3 |
| Example 5 | 1.0 | 3 |
| Example 6 | 4.0 | 2 |
| Example 7 | 5.0 | 6 |
| Example 8 | 0.73 | 2 |
| Example 9 | 4.0 | 8 |

These results indicate that compounds of the invention have potent antagonist or inverse agonist activity at the H3 receptor, both in terms of binding and in terms of inhibition of the functional response caused by receptor activation. The compounds tested above exhibit IC₅₀ values significantly less than 1 μM, with the compounds showing low nanomolar affinity at the H3 receptor. Accordingly, the compounds of the invention are expected to have usefulness in the prevention or treatment of conditions, such as those discussed above, in which H3 receptor activity is implicated.

In addition, the compounds of the present invention may possess variously advantageous pharmacological and/or toxicological profiles, when tested in a variety of standard tests for such parameters. For example, the compounds of the invention may exhibit one or more potentially useful properties for in vivo use, when characterised by pharmacological and/or toxicological tests including: HERG interaction (which is an indication of potential cardiotoxicity, and measures the effects of the compounds on the human ether-a-go-go-related gene, using for example the PatchXpress 7000A platform); CypP₄₅₀ interactions (which may be measured in accordance with the FDA draft guidelines for drug interaction studies (study design, data analysis and implications for dosing and labeling) (September 2006), see www.fda.gov); phototoxicity (for example using a protocol in accordance with assay details outlined in the OECD guidelines for testing of chemicals: 432 In Vitro 3T3 Neutral Red Uptake phototoxicity test, April 2004); determination of pharmacokinetic parameters (for example following in vivo dosing via multiple routes, with plasma concentrations of compounds being determined from venous blood samples using an LC-MS/MS protocol); and in vivo receptor occupancy (determined, for example, using protocols based on Medhurst et al., Journal of Pharmacology and Experimental Therapeutics, 2007, 321, 1032). These standard tests for the characterisation of drug molecules are well known to the skilled person.

### REFERENCES

1. J.-M. Arrang, M. Garbarg and J.-C. Schwartz. Nature, 1983, 302, 832
2. T. W. Lovenberg, B. L. Roland, S. J. Wilson, X. Jiang, J. Pyati, A. Huvar, M. R. Jackson and M. G. Erlander. Mol. Pharmacol., 1999, 55, 1101.
3. S. J. Hill, C. Ganellin, H. Timmermans, J. C. Schwartz, N. Shankley, J. M. Young, W. Schunack, R. Levi and H. L. Haas. Pharmacol. Rev., 1997, 49, 253.
4. Passani M B, Lin J-S, Hancock A, Crochet S, Blandina P. The histamine H3 receptor as a novel therapeutic target for cognitive and sleep disorders. Trends Pharmacol. Sci. 2004; 25:618-25.
5. Witkin J M, Nelson D L. Selective histamine H3 receptor antagonists for treatment of cognitive deficiencies and other disorders of the central nervous system. Pharmacol. Ther. 2004; 103:1-20
6. Monti J. M et al. Effect of Selective activation or blockade of the hitamine H3 receptor on sleep and wakefulness. 1991 Eur. J. Pharmacol. 205, 283-287.
7. Esbenshade T. A. et al. Biochemical Pharmacology 68 (2004) 933-945.
8. Morimoto T, Yamamoto Y, Yamatodani A. Leptin facilitates histamine release from the hypothalamus in rats. Brain Res. 2000; 868:367-9
9. A. A. Hancock. Biochem. Pharmacol., 2006, 71, 1103.
10. A. A. Hancock and M. E. Brune. Expert Opin. Investig. Drugs, 2005, 14, 223
11. D. Farzin, L. Asghari and M. Nowrouzi. Pharmacol. Biochem. Behav., 2002, 72, 751.
12. WO 04/089410
13. Medhurst A. D. et al. Biochemical Pharmacology 73 (2007) 1182-94
14. Esbenshade T. A et al. J. Pharmacol. Exp. Ther. 2005 313(1) 165-75

1. A compound of the formula: 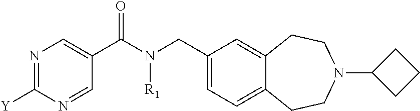
wherein:
R₁ represents C₁₋₆ alkyl or H;
Y represents —NR₂R₃ as depicted in formula (B), or a ring of formula (A) 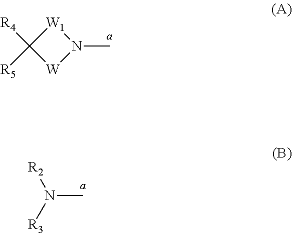
wherein ^{a} represents the point of attachment to the pyrimidinyl ring;
R₂ represents C₁₋₄alkyl substituted by C₁₋₃ alkoxy;
R₃ represents C₁₋₄ alkyl;
W represents —(CH₂)ₙ—;
W₁ represents —(CH₂)ₚ—;
n represents 1 or 2 or 3;
p represents 1 or 2;
R₄ represents C₁₋₄ alkoxy, C₁₋₆ alkyl or halogen; and
R₅ represents halogen or H,
provided that, when R₄ represents halogen, R₅ is not H,
or a pharmaceutically acceptable salt thereof.

2. A compound according to claim 1 wherein Y represents a ring of formula (A).

3. A compound according to claim 1 wherein p is 1.

4. A compound according to claim 1 wherein R₁ is H, n is 2, p is 1, R₄ is methoxy and R₅ is H.

5. A compound according to claim 1 wherein R₁ is H, n is 1, p is 1, R₄ is methoxy and R₅ is H.

6. A compound according to claim 1 wherein R₄ represents C₁₋₄ alkoxy.

7. A compound according to claim 1 which is:
N-[(3-cyclobutyl-2,3,4,5-tetrahydro-1H-3-benzazepin-7-yl)methyl]-2-[(3R)-3-methoxypyrrolidin-1-yl]pyrimidine-5-carboxamide;
N-[(3-cyclobutyl-2,3,4,5-tetrahydro-1H-3-benzazepin-7-yl)methyl]-2-[(3S)-3-methoxypyrrolidin-1-yl]pyrimidine-5-carboxamide;
N-((3-cyclobutyl-2,3,4,5-tetrahydro-1H-3-benzazepin-7-yl)methyl)-2-(3-methoxyazetidin-1-yl)pyrimidine-5-carboxamide;
N-[(3-cyclobutyl-2,3,4,5-tetrahydro-1H-3-benzazepin-7-yl)methyl]-2-(3,3-difluoropyrrolidin-1-yl)pyrimidine-5-carboxamide;
N-((3-cyclobutyl-2,3,4,5-tetrahydro-1H-3-benzazepin-7-yl)methyl)-2-(4,4-difluoropiperidin-1-yl)pyrimidine-5-carboxamide;
N-[(3-cyclobutyl-2,3,4,5-tetrahydro-1H-3-benzazepin-7-yl)methyl]-2-[(3S)-3-methoxypyrrolidin-1-yl]-N-methylpyrimidine-5-carboxamide;
N-[(3-cyclobutyl-2,3,4,5-tetrahydro-1H-3-benzazepin-7-yl)methyl]-2-[(3R)-3-methoxypyrrolidin-1-yl]-N-methylpyrimidine-5-carboxamide;
N-[(3-cyclobutyl-2,3,4,5-tetrahydro-1H-3-benzazepin-7-yl)methyl]-2-{[(2S)-2-methoxypropyl](methyl)amino}pyrimidine-5-carboxamide; or
N-[(3-cyclobutyl-2,3,4,5-tetrahydro-1H-3-benzazepin-7-yl)methyl]-2-{[(2R)-2-methoxypropyl](methyl)amino}pyrimidine-5-carboxamide,
or a pharmaceutically acceptable salt thereof.

8. A pharmaceutical composition comprising a compound according to claim 1, together with one or more pharmaceutically acceptable excipients.

9. A composition according to claim 8, comprising one or more additional, pharmaceutically active ingredients.

10. (canceled)

11. (canceled)

12. A method of treatment or prevention of a condition whose development or symptoms are linked to histamine H3 receptor activity, the method comprising the administration, to a subject in need of such treatment or prevention, of a therapeutically effective amount of a compound according to claim 1.

13. A method according to claim 12, wherein the condition is a disorder of the central nervous system.

14. A method according to claim 12, wherein the condition is a disorder selected from schizophrenia, neurodegenerative disorders (such as Alzheimer's Disease), cognitive disorders (such as dementia and schizophrenia), sleep disorders (such as narcolepsy and hypersomnia), pain, obesity, attentional disorders and epilepsy.

15. (canceled)

16. A method for preparing a compound according to claim 1, the method comprising the step of reacting an intermediate having the formula: 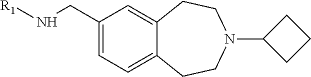
wherein R₁ is H or C₁₋₆ alkyl;
with a pyrimidine derivative of the formula: 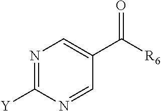
wherein Y represents —NR₂R₃ as depicted in formula (B), or a ring of formula (A) 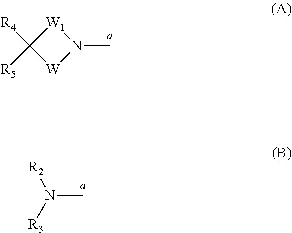
wherein ^{a} represents the point of attachment to the pyrimidinyl ring;
R₂ represents C₁₋₄alkyl substituted by C₁₋₃ alkoxy;
R₃ represents C₁₋₄ alkyl;
W represents —(CH₂)ₙ—;
W₁ represents —(CH₂)ₚ—;
n represents 1 or 2 or 3;
p represents 1 or 2;
R₄ represents C₁₋₄ alkoxy, C₁₋₆ alkyl or halogen; and
R₅ represents halogen or H,
provided that, when R₄ represents halogen, R₅ is not H; and
R₆ is OH or a carbonyl activating group.

17. A method according to claim 16, wherein R₆ is OH or OR₇, R₇ being a carboxyl activating group.

18. A compound of formula (i) or (ii): 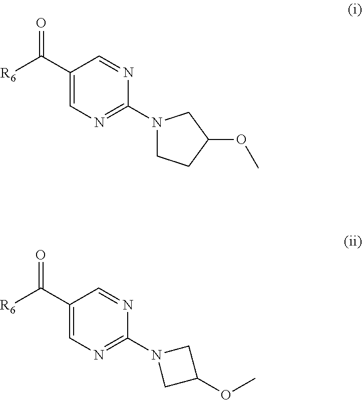
wherein R₆ is OH or a carbonyl activating group.
